(19) United States
(12) Patent Application Publication
(10) Pub. No.: US 2005/0026262 A1
(43) Pub. Date: Feb. 3, 2005

(54) Sonication-enhanced digestion process

(75) Inventors: Jun Yoshitani, West Chicago, IL (US); Donald O. Johnson, Naperville, IL (US); Michael L. Wilkey, Oak Park, IL (US)

Correspondence Address: Heidi A. Dare;Brinks Hofer GIlson & Lione, P.O. Box 10395, Chicago, IL, 60610, US

(73) Assignee: SonoEnergy, LLC

(21) Appl. No.: 10/630,294

(22) Filed: Jul. 30, 2003

(51) Int. Cl. C12P 1/00; C12N 1/12; C12N 1/20

(52) U.S. Cl. 435/167; 435/252.1

(57) ABSTRACT

A system and method are provided for sonication-enhanced digestion of cellular matter. The method includes supplying cellular matter to a bioreactor, sonically disrupting the cellular matter, and providing the disrupted cellular matter for microbial digestion in the bioreactor. The system includes a bioreactor for cellular matter, the bioreactor having an inlet and an outlet; a sonic energy source connected to the bioreactor; and at least one rotating member connected to the bioreactor. The cellular matter enters the bioreactor through the inlet, is mixed by the rotating member and is subjected to sonic energy and microbial digestion in the bioreactor.

### BACKGROUND

1. Technical Field

The present invention relates to digestion of cellular matter. In particular, the present invention relates to sonication-enhanced digestion of cellular matter to increase biogas and biofuel production.

2. Background Information

Biomass is organic matter that may be used as an energy source. Biomass is derived from sources such as agricultural and municipal wastes. Agricultural and municipal wastes represent a largely, as yet, untapped potential energy sources. For example, biogas, a byproduct of anaerobic digestion of waste, represents a potentially important energy resource. Biogas may be generated from sources such as manure and dedicated energy crops using anaerobic digestion. Additionally, biofuel, such as ethanol, may be generated from carbohydrates present in waste, such as lignocellulose. Complex organic polymers present in the waste are hydrolyzed into smaller polymer subunits, such as monomers, by the addition of water and or the digestion mediated by microorganisms. In ethanol production, the sugars resulting from the hydrolysis are then fermented, distilled and purified into useable biofuel.

As a microbial process, anaerobic digestion is a net energy producer. The net energy yields associated with anaerobic digestion make it an attractive treatment option for the production of energy from biomass and wastes. However, the main disadvantages of anaerobic digestion of biomass and wastes to produce energy are the long hydraulic retention times and the large reactor volumes. Both the long hydraulic retention time, typically over 20 days, and the large reactor volumes add considerable cost to producing energy from biomass and wastes.

In addition, despite the long hydraulic retention time, digestion of the waste is still incomplete, leaving a portion of the biomass and wastes unable to be used for production of energy. The complex organic polymers present in the biomass and wastes are difficult to degrade. For example, agricultural waste contains a high percentage of lignocellulose. Lignocellulose includes hemicellulose, lignin, and cellulose. The structure of these molecules, such as cellulose, having long chain glucose molecules with beta-1,4 linkages, make the molecules resistant to degradation by microorganisms.

Currently, there are three basic techniques for converting lignocellulosic biomass, including cellulose, hemicellulose, and lignin, into fermentable simple sugar solutions which can be used for energy. The techniques include a one-step acid hydrolysis in which the hemicellulose and cellulose are broken down in a single step using concentrated aqueous solutions of strong mineral acids. Drawbacks of this technique include essential recovery processes for the acid based on economic and environmental issues, high quality equipment for exposure to the acids, and some sugar degradation due to the unequal hydrolysis times required for hemicellulose and cellulose.

A second technique is a two-step dilute acid process in which the hemicellulose and cellulose parts are hydrolyzed separately. However, due to a much higher temperature requirement in the second step (around 200° C.) considerable amounts of both sugar and lignin degradation products are formed.

In the third technique an enzymatic process is used in which the lignocellulosic biomass is first pretreated in order to increase accessibility for the cellulolytic enzymes. The enzymatic process is also a two-step hydrolysis technique although the cellulose fraction is broken down using cellulases instead of acids. The milder conditions of the enzymatic process result in fewer by-products being liberated, however, the cost of the treatment makes the process unlikely to be used for large-scale conversion of biomass to energy.

Similar to the problems with obtaining cost efficient and safe energy from lignocellulose, the use of anaerobic digestion to produce biogas for energy has problems for cost effective, efficient production of energy. The anaerobic digestion technology currently being applied for agricultural waste is inherently inefficient. The prevalent anaerobic processes; commonly known as “Plug Flow” and “Complete Mix”, do little to create more optimal growing conditions for the microbial population responsible for facilitating anaerobic digestion. Although microbial growth is enhanced, a large percentage of the waste remains undigested and therefore unavailable for energy production using the current anaerobic processes.

The present invention addresses the deficiencies in the current anaerobic digestion processes and lignocellulosic hydrolysis processes. The present invention provides an improved system and method for enhancing digestion or hydrolysis of cellular matter and producing increased amounts of biogas and biofuel, respectively, when compared to a similar amount of input biomass and wastes digested using conventional technologies. The problems of long hydraulic retention time and incomplete digestion are solved by the present invention leading to increased, cost effective biogas and biofuel production.

### BRIEF SUMMARY

In order to alleviate one or more shortcomings of the prior art, a sonication-enhanced digestion method and system are provided herein.

According to one aspect of the present invention, there is provided a method for sonication-enhanced digestion of cellular matter. The method comprises supplying cellular matter comprising microbes to a bioreactor, sonically disrupting the cellular matter, and mixing the cellular matter in the bioreactor for a time sufficient for the cellular matter to be digested.

In another aspect of the present invention, a method for sonication-enhanced degradation of cellular matter is provided. The method comprises supplying cellular matter comprising microbes to a first bioreactor and subjecting the cellular matter to sonic energy in a frequency range of about 1 kHz to about 10 kHz in the first bioreactor.

In another aspect of the present invention, a system for sonication-enhanced digestion of cellular matter is provided. The system comprises a bioreactor for cellular matter, the bioreactor having an inlet and an outlet, a sonic energy source operatively connected to the bioreactor, and at least one rotating member operatively connected to the bioreactor. The cellular matter enters the bioreactor through the inlet, is mixed by the at least one rotating member and is subjected to sonic energy and microbial digestion in the bioreactor.

In another aspect of the present invention, an apparatus for sonication-enhanced degradation of cellular matter is provided. The apparatus comprises a first bioreactor, and a sonic energy source operatively connected to the first bioreactor. The first sonic energy source subjects the cellular matter to sonic energy in a frequency range of about 1 kHz to about 10 kHz.

In another aspect of the present invention, a system for sonication-enhanced digestion of cellular matter is provided. The system comprises a bioreactor for cellular matter, means for sonically disrupting cellular matter contained in the bioreactor, and means for mixing the cellular matter in the bioreactor. The cellular matter is sonically disrupted, mixed, and digested in the bioreactor.

Advantages of the present invention will become more apparent to those skilled in the art from the following description of the preferred embodiments of the present invention that have been shown and described by way of illustration. As will be realized, the invention is capable of other and different embodiments, and its details are capable of modification in various respects. Accordingly, the drawings and description are to be regarded as illustrative in nature and not as restrictive.

### BRIEF DESCRIPTION OF THE DRAWINGS

FIG. 1A is a block diagram of a sonication-enhanced digestion system for an embodiment of the present invention;

FIG. 1B is a block diagram of a sonication-enhanced digestion system for an embodiment of the present invention;

FIG. 2 is a schematic diagram of a sonication-enhanced digestion system for an embodiment of the present invention;

FIG. 3 is a cross sectional view of the digestion system shown in FIG. 2;

FIG. 4 is a partial view of the rotating member of the bioreactor of the present invention shown in FIG. 2; and

FIG. 5 is a schematic diagram of a sonication system for an alternative embodiment of the present invention.

### DETAILED DESCRIPTION OF THE DRAWINGS AND THE PRESENTLY PREFERRED EMBODIMENTS

The present invention provides improved methods and systems for digesting cellular matter and increasing biogas and biofuel production. Cellular matter as used herein refers to agricultural materials, such as manure, residues, and dedicated energy crops, and municipal waste such as sewage sludge and solid waste, industrial waste, such as food processing waste, and other types of cellular matter known to one of skill in the art to produce energy. Biogas includes, but is not limited to, methane, carbon dioxide, and hydrogen sulfide. Biofuel includes, but is not limited to, ethanol, methanol, and biodiesel.

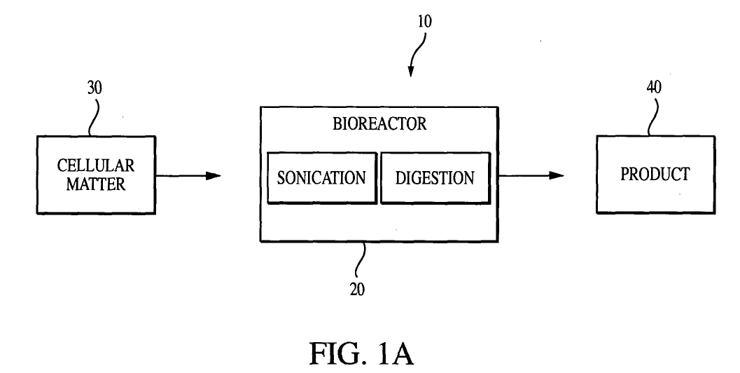

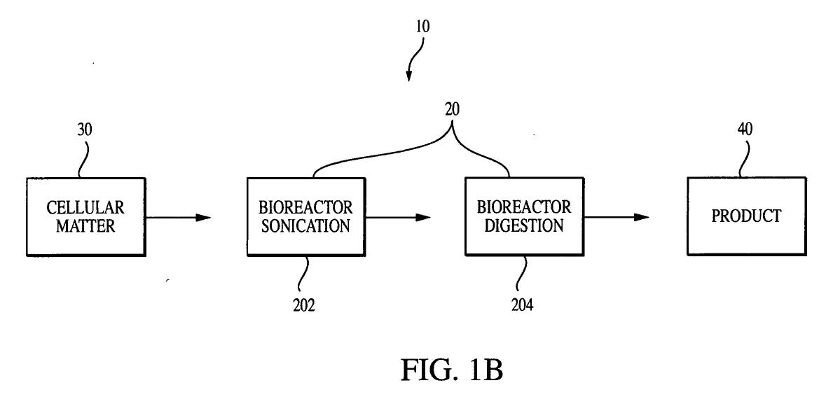

FIG. 1A illustrates a block diagram of an embodiment of a sonication-enhanced digestion system 10 and method of the present invention. The system 10 comprises a bioreactor 20 for sonication and digestion of cellular matter 30. As shown in FIG. 1A, cellular matter 30 enters the bioreactor 20, is subjected to sonication and digestion in the bioreactor 20 and a product 40 exits the bioreactor. The product 40 may be in the form of a liquid, gas, or solid, or any combination thereof. Alternatively, the system 10 comprises a plurality of bioreactors 20 for the sonication and digestion of the cellular matter 30. In an embodiment shown in FIG. 1B, the cellular matter 30 enters a bioreactor 202. The cellular matter 30 is subjected to sonication and digestion in the bioreactor 202. The cellular matter 30 exits the bioreactor 202 and enters a bioreactor 204. In the bioreactor 204, the cellular matter 30 is subjected to digestion. In addition, the cellular matter 30 may be subjected to sonic energy in the bioreactor 204. The product 40 exits the bioreactor 204. Additional embodiments may comprise a plurality of bioreactors 20 for sonication and digestion of cellular matter 30, for example, the plurality of bioreactors 20 may each represent a zone as discussed below. The sonication and digestion conditions for each bioreactor may be different with respect to each of the other plurality of bioreactors.

In an embodiment of the present invention, a method is provided for sonication-enhanced digestion of cellular matter. The method as described herein may be used with a system comprising a bioreactor or a plurality of bioreactors. The method comprises adding cellular matter to a bioreactor through an inlet at a first end of the bioreactor. The cellular matter is subjected to sonication in the bioreactor using a sonic energy source for a sufficient amount of time to disrupt suspended solid particles in the cellular matter as well as disrupt and breakdown the cellular matter into smaller subunits. As used herein, disruption may also include disintegration, fragmentation, and breaking apart of organic polymers and cellular membranes, as well as other forces known to those of skill in the art. Following sonication, the cellular matter comprising smaller subunits may be microbially digested for a time sufficient to anaerobically digest the cellular matter and produce biogas and biofuel. The biogas and biofuel produced may be removed through an outlet at a second end of the bioreactor. The method may include forming the biogas under a vacuum formed in the bioreactor by removing the biogas from the bioreactor.

In an embodiment, the cellular matter may be sonicated and microbially digested in the same bioreactor. Alternatively, the cellular matter may be transferred after sonication to at least one additional bioreactor for subsequent microbial digestion and/or sonication.

In an embodiment, the method may further comprise mixing, folding, and advancing the cellular matter from the first end of the bioreactor to the second end with a rotating member operatively connected to the bioreactor. Additionally, the method may comprise adding conditioned microbes to the cellular matter in the bioreactor. Adding the cellular matter to the bioreactor may comprise adding manure, lignocellulose, or municipal waste.

The method provided herein subjects the cellular matter to sonic energy in the bioreactor. The term sonication as used herein refers to the application of sound waves (acoustic energy) transmitted through a liquid medium (manure, water, oil, etc.) as a wave of alternating cycles of compression and rarefaction. The waves cause particles to oscillate about their mean position. When sound waves are in compression phase, positive pressure is exerted on the liquid and pushes molecules together, while in rarefaction, negative pressure is exerted and pulls molecules apart. As the vibrational energy is increased, the pressure in the liquid falls below its vapor pressure creating voids or cavities called microbubbles. The phenomenon is called cavitation. After formation, the microbubbles grow in size until a maximum negative pressure is reached. In the succeeding compression cycle, the bubbles are forced to contract and implode, releasing large amounts of energy within a micro-millimeter of the bubble. Temperatures on the order of 5,0000° K., and pressures of 500 to 1,500 atm have been experimentally determined to occur at the collapsing interface during bubble implosion.

At low frequency, a significant part of the energy released causes shock waves, hydro-jets, and hydro-shear in the liquid medium. When impacted by these forces, suspended solid particles in the liquid medium disintegrate into smaller particles. The influence of shock waves, hydro-jets, and hydro-shear is, however, limited to solid particles situated in close proximity of the acoustic energy. The distance of influence depends on many factors, such as the frequency used in sonication, power levels used in sonication, size and shape of the reactor, and density (specific gravity) of the cellular matter and liquid medium.

Providing the sonic energy to the cellular matter comprises supplying energy from a power source connected to a wave-form generator connected to a transducer that is connected to a contact plate on the exterior of the bioreactor. The wave-form generator, connected to the transducer, may also be provided to monitor the sonication energy provided to the bioreactor using an oscilloscope. The power source supplies electric power to the transducer. The power supplied to the transducer varies based on the amount and type of cellular matter in the bioreactor and the amount of biogas and/or biofuel that may be produced without application of sonic energy in comparison to the amount of increased biogas and/or biofuel production that would result with the application of the sonic energy. One skilled in the art will recognize that the amount of power supplied to the transducer may be optimized to have a net energy production from the resulting biogas production with respect to the input energy.

The transducer converts electrical power supplied from the power generator to vibrational acoustic energy. A plurality of transducers may be connected to the contact plate to provide sonic energy to the cellular matter. The transducer as used in the present invention may be magnetostrictive, for example, using a stack of ferromagnetic crystals, or electrostrictive, using piezoelectric ceramics. When the power source is activated, the energy supplied to the transducer is converted by the transducer into vibrational acoustic energy. The vibrational acoustic energy is transmitted through the contact plate, through the bioreactor wall and into the interior of the bioreactor. The contact plate as used herein is similar to the horn or tool in a sonication system known to one of skill in the art, for example, a sonication system used for welding. The distance the vibrational acoustic energy travels into the bioreactor depends on the frequency, power, liquid and cellular matter density, and reactor size and shape, as discussed above. The cellular matter in the bioreactor in close proximity to the contact plate is disrupted by the supply of sonic energy. The term close proximity as used herein means the zone in which the cellular matter is subjected to sonic energy sufficient to disintegrate, disrupt and breakdown the cellular matter.

For example, in an embodiment, the zone in which disruption of the cellular matter occurs may be within about 0.01 to about 10 inches from the wall of the bioreactor adjacent to the contact plate. However, other distances are possible for the disruption of the cellular matter. In an embodiment of the present invention, the sonic energy supplied to the first end of the bioreactor is in the frequency range from about 1 kHz to about 10 kHz, more preferably in the frequency range from about 2 kHz to about 7 kHz.

Microbially digesting the cellular matter, that has been disrupted by sonication, comprises digesting the cellular matter with microbes for a time sufficient to anaerobically digest the cellular matter and produce biogas, such as methane and carbon dioxide. The cellular matter added to the bioreactor typically contains microbes capable of digesting the cellular matter. The term microbes as used herein includes bacteria, archaea, fungi, protozoa, and other microorganisms known to one of skill in the art to digest the cellular matter to produce biogas. Exogenous microbes do not need to be added to the cellular matter for digestion, although exogenous microbes such as bacteria may be added. In an embodiment of the present invention, discussed below, conditioned microbes from the cellular matter are added back to the cellular matter during digestion. Some of the microbes present in the cellular matter may be killed by disruption in the sonication process when the microbes are in close proximity to the sonic energy source and a frequency range of about 1 kHz to about 10 kHz is supplied. However, a population of the microbes will survive and microbial growth will be enhanced by the disruption of complex polymers in the cellular matter and subsequent provision of substrates for microbial digestion.

Methods of producing biogas using anaerobic digestion of sewage sludge are known to one of skill in the art. Anaerobic digestion refers to the production of biogas, including methane, carbon dioxide, and hydrogen sulfide, and other gases in trace amounts, from cellular matter by microbial digestion. As described above, anaerobic digestion by itself is inherently inefficient. However, increased production of biogas by anaerobic digestion occurs when the cellular matter is subjected to sonic energy prior to anaerobic digestion. The cellular matter that has been broken down by sonic energy into smaller subunits may then be more efficiently digested by microbes present in the cellular matter as compared to digestion alone. Digestion of the cellular matter to produce biogas includes digestion by acid-forming microbes and methanogenic microbes, as well as other microbes. Many types of acid-forming and methanogenic microbes are present in the cellular matter and digest a variety of substrates. Acid-forming microbes form acetate, long-chain fatty acids, carbon dioxide, H₂, NH₂, and HS⁻. Methanogenic microbes produce methane and carbon dioxide.

In the first step of the digestion process, polymeric substrates such as polysaccharides, proteins, and lipids are hydrolyzed into smaller subunits. In the second step, the hydrolyzed compounds are fermented to produce acetate, long-chain fatty acids, CO₂, H₂, NH₄ and HS⁻. In a parallel step, proton-reducing acetogenic microbes (syntrophic organisms) degrade propionate, long-chain fatty acids, alcohols, amino acids, and aromatic compounds to H₂, and acetate. Degradation of these compounds with production of H₂ sometimes upsets the anaerobic digestion process unless the concentration of H₂ is maintained low by H₂ utilizing methanogenic microbes. Thus, the third step involves two different groups of methanogens, the hydrogenotrophic methanogens that use the H₂ produced by other microbes to reduce CO₂ to CH₄, and the acetotrophic methanogens that metabolize acetate to form CO₂ and CH₄.

In an embodiment of the present invention, the cellular matter may progress through zones of disruption, acid formation, and methane formation as the cellular matter is mixed, folded and advanced through the bioreactor. While three zones are described below for the preferred embodiment, additional zones, fewer zones and alternative types of zones are possible. In addition, the zones as described herein may be overlapping as well as discrete zones. The zones may be present in one bioreactor or alternatively, the zones may be present in more than one bioreactor operatively linked in series. For example, each zone may be present in a separate bioreactor.

In a first zone, a hydrolysis zone, subjecting the cellular matter to sonic energy occurs. The frequency of the sonic energy applied to the cellular matter in the hydrolysis zone may be in the frequency range of about 1 kHz to about 10 kHz. The complex cellular matter is disintegrated, disrupted and broken down into simpler, smaller compounds. In a second zone, the acid zone, microbial digestion occurs wherein acid-forming microbes, including acetogenic microbes, present in the cellular matter, begin to breakdown the polymers and smaller subunits as soon as they are formed after subjection of the cellular matter to sonic energy. As more, smaller subunits become available, the number of acid-forming microbes present in the cellular matter multiply so that the acid zone becomes conditioned with a higher density of acid-forming microbes in a section of the acid zone toward the second end of the bioreactor. In an embodiment of the present invention, a portion of the conditioned cellular matter, having an increased density of acid-forming microbes, may be added back to the newly added cellular matter near the beginning of the acid zone.

In a third zone of the bioreactor, the methane zone, another population of microbes known generally as methanogenic microbes present in the cellular matter further degrade products formed in the acid zone to produce biogas, including methane and/or carbon dioxide. Similar to the acid zone, the methane zone becomes conditioned with a higher density of methanogenic microbes in a section of the methane zone near the second end of the bioreactor. A portion of the conditioned cellular matter, containing an increased population of methanogenic microbes, may be added back to the beginning of the methane zone to increase the production of biogas. In addition, in the methane zone, sonic energy may be applied to the cellular matter in an amount sufficient to disintegrate solids and disrupt microbes present with the cellular matter.

The amount of conditioned cellular matter containing an increased population of microbes, referred to herein as conditioned microbes, added back to each zone will depend on the type and concentration of the cellular matter being digested. The amount of conditioned microbes added back to the beginning of each zone will sufficiently enhance the degradation by the existing microbes to increase the amount of desired product, for example, an increased amount of biogas. Conditioned microbes may be added back to a single zone or more than one zone or conditioned microbes need not be added to any zone.

In an alternative embodiment, exogenous microbes, such as bacteria, may be supplied to any of the zones encompassing microbial degradation. The amount of exogenous microbes added will depend on the type and concentration of the cellular matter being digested and the amount of resulting product desired. The amount of exogenous microbes added will be sufficient to enhance production of the desired product in comparison to the production of the product without the addition of exogenous microbes.

In an embodiment of the method of the present invention, the microbes present in the bioreactor may be continually used in the respective zones as long as new cellular matter is added to provide new substrate on which the microbes may continue to grow. Alternatively, the cellular matter may be digested in a batch-wise manner wherein an amount of cellular matter is supplied to the bioreactor, subjected to sonic energy, microbial digestion, and removed from the bioreactor before new cellular matter is added.

In an alternative embodiment of the present invention, sonic energy may be applied to the cellular matter in any zone, including the acid zone and/or the methane zone. The sonic energy is applied to the acid zone and the methane zone using a sonic energy source as described above. The frequency range for the sonic energy supplied to the hydrolysis zone, as described above, is in the frequency range from about 1 kHz to about 10 kHz. The frequency range for the sonic energy supplied to the acid zone and the methane zone, and any additional zone, is in the frequency range from about 1 kHz to about 2,000 kHz.

In an embodiment of the present invention, the method further comprises mixing, folding, and advancing the cellular matter from the first end of the bioreactor to the second end using a rotating member operatively connected to the bioreactor. The term mixing as used herein to disperse the cellular matter may be multi-directional, including forward, reverse, inward, outward and advancing motions. Mixing of the cellular matter generally moves the cellular matter axially from the first end to the second end of the bioreactor. In addition, a portion of the cellular matter moves vertically and axially toward the first end of the bioreactor by mixing. Thus, a portion of the cellular matter mixes within a zone and a portion of the cellular matter moves axially toward the second end of the bioreactor. The motion of the rotating member for mixing as used herein includes rotating, circular, vibrational, oscillating, and sweeping motions, as well as other motions known by those of skill in the art to mix, fold, and advance the cellular matter in the bioreactor.

Mixing of the cellular matter may be intermittent or continuous. In addition, the number of rotations per minute (rpm) of the rotating member may vary and are adjustable depending on the requirements of the particular cellular matter, including considerations such as a consistency in the percentage of the fiber within the cellular matter. The rotating member may rotate from about 0.25 to about 5.0 rpm, more preferably from about 0.5 to about 3.0 rpm, to mix, fold, and advance the cellular matter from the first end to the second end of the bioreactor.

The method further comprises removing the biogas formed in the methane zone and forming a vacuum in the bioreactor by removing the biogas. The biogas may be removed using a pump to draw off the gas formed. Removing the biogas may be performed by any technique commonly known in the art for removing gas from a reactor and maintaining a vacuum. The method further comprises intermittently removing accumulation of inorganic solids and debris from the bioreactor.

The method may further comprise providing a heating means for a portion of the bioreactor. The heating means may be provided on the exterior of the bioreactor for the portion of the reactor in which the microbial digestion occurs, for example the acid zone and the methane zone. Providing a heating means allows the methanogenic degradation to occur at thermophilic temperatures. The term thermophilic as used herein describes temperatures in the range of about 50° C. to about 60° C.

In an embodiment of the present invention, a system is provided for sonication-enhanced digestion of cellular matter. The system comprises a bioreactor having an inlet at a first end and an outlet at a second end. The system further comprises a sonic energy source operatively connected to the bioreactor to supply sonic energy to at least one zone within the bioreactor. The sonic energy source further comprises a power supply, a wave-form generator, a transducer, and a contact plate. The sonic energy source supplies the sonic energy to the at least one zone as described above.

The system, in an embodiment, further comprises at least one rotating member to mix, fold, and advance the cellular matter in the bioreactor from the first end of the bioreactor to the second end of the bioreactor as described above. The at least one rotating member rotates about a shaft operatively connected to the bioreactor. The shaft is driven by an electric motor. The electric motor provides a variable frequency drive, although a constant frequency drive may also be used to drive the shaft.

The system may further comprise a means for supplying conditioned microbes from an end of a zone of digestion to a beginning of the zone of digestion. The conditioned microbes are described above.

The system further comprises a gas exhaust valve operatively connected to the bioreactor. The valve may be connected to a pump to draw off the biogas formed in the bioreactor thereby creating a vacuum in a headspace formed in the bioreactor. The system may further comprise a heating means for supplying heat to a portion of the bioreactor. In an embodiment of the present invention, the second end of the bioreactor is elevated with respect to the first end. The degree of elevation is adjustable.

In another embodiment of the present invention, the system comprises a plurality of bioreactors. A first bioreactor is operatively connected to at least one more bioreactor. The first bioreactor comprises a first sonic energy source that supplies sonic energy to the frequency range of about 1 kHz to about 10 kHz. The sonic energy source is described above. The first bioreactor may further comprise at least one rotating member and a process controller, as described above.

The embodiment may further comprise at least one more bioreactor connected to the first reactor. The connection between the first bioreactor and the at least one more bioreactor may be a physical connection, or alternatively, the connection may comprise the transfer of cellular matter from the first bioreactor to the at least one more bioreactor without physical contact between the bioreactors. The at least one more bioreactor may comprise at least one more sonic energy source for subjecting cellular matter to sonic energy at a frequency range of about 1 kHz to about 2,000 kHz.

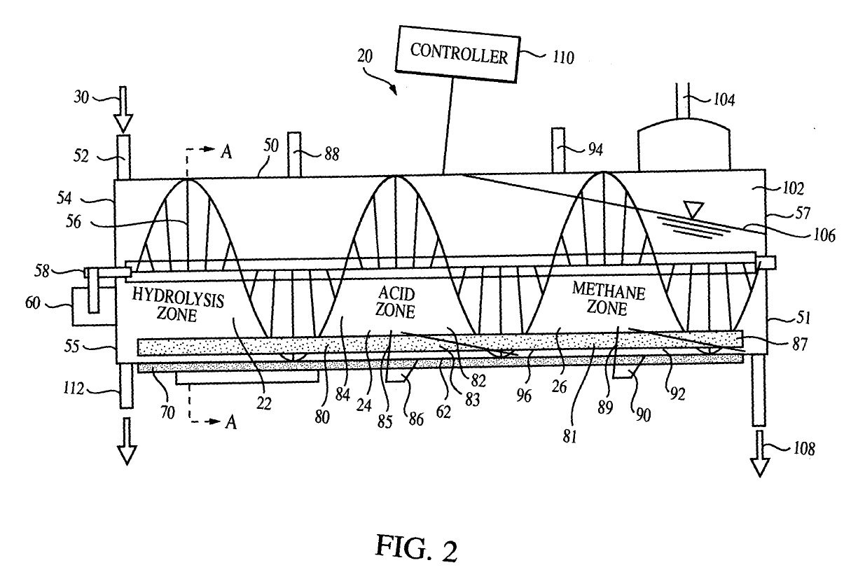

Detailed descriptions of preferred embodiments of the system of the present invention are provided below and illustrated in FIGS. 2-5. The detailed descriptions provided herein are in connection with a preferred embodiment and are not meant to be limiting.

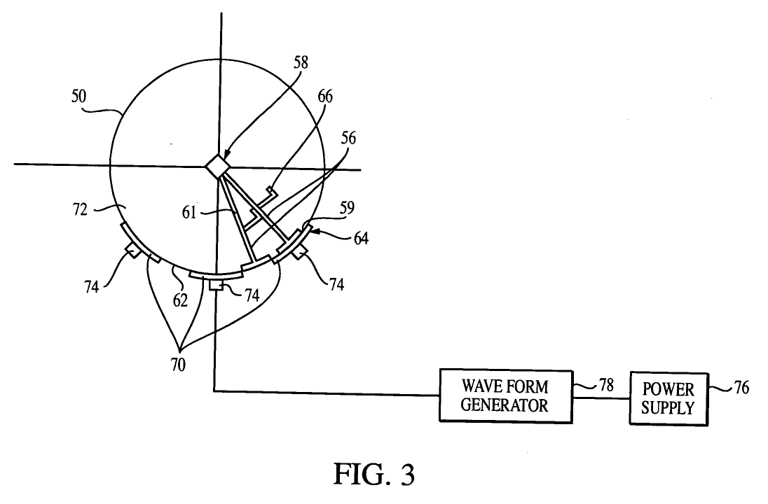

FIGS. 2 and 3 illustrate the bioreactor 20 of a preferred embodiment of the present invention. The bioreactor 20 comprises an elongated tank 50. The tank 50 in the preferred embodiment of the present invention is a horizontally elongated tank having a circular cross section. The diameter of the tank 50 ranges from about 6 feet to about 12 feet, and the length of the tank is from about 25 feet to about 75 feet. The capacity of the tank 50 is about 10,000 to about 30,000 gallons. The tank 50 further comprises a removable end plate 55 on the first end 54 and a removable plate 51 on a second end 57. The tank 50 may be constructed as a single piece, or alternatively, the tank 50 may be constructed with modular construction.

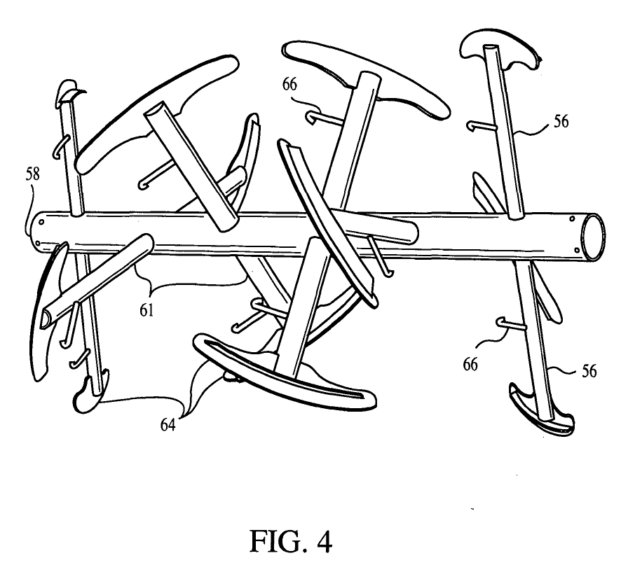

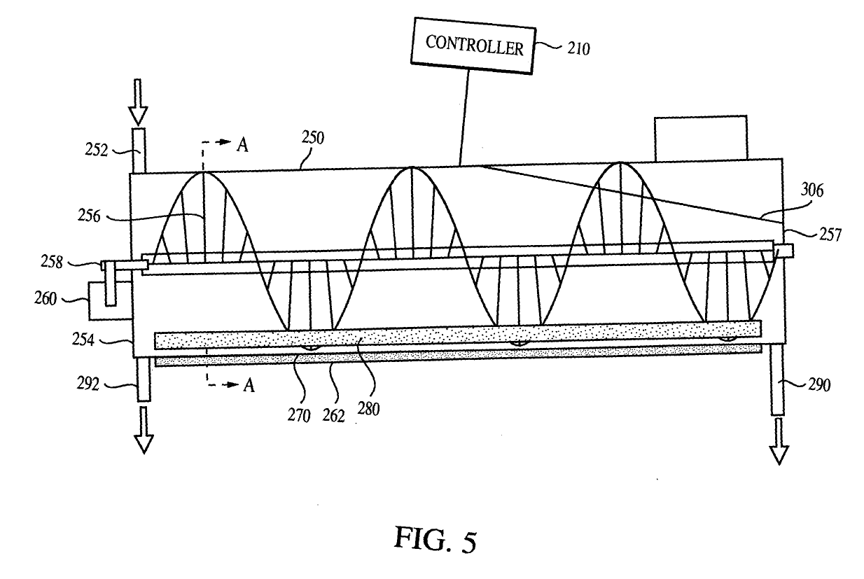

As shown in FIG. 2, the cellular matter 30 enters the bioreactor 20 through an inlet 52 at a first end 54 of the bioreactor 20. The cellular matter 30 is mixed, folded, and advanced in the bioreactor by at least one rotating member 56. The rotating member 56 is shown in a cross-sectional view in FIG. 3 and in FIGS. 4 and 5. In a preferred embodiment shown in FIG. 2, the rotating member 56 rotates about a center shaft 58. The center shaft 58 is supported at the ends and at intermediate points and extends the length of the tank 50 from the first end 54 to the second end 57. The shaft 58 is driven by an electric motor 60. The rotating member 56 disburses a cellular matter 30 such that the distribution of the cellular matter 30 within the tank 50 may become substantially uniform. A plurality of rotating members 56 may be used for disbursement of the cellular matter 30.

As shown in FIGS. 2 and 3, a plurality of rotating members 56 mix and fold the cellular matter 30 to disburse and advance the cellular matter 30 within the tank 50. In a preferred embodiment, shown in FIG. 4, the central shaft 58 has a plurality of rotating members 56 extending radially from the shaft 58 and the plurality of rotating members 56 form a helical pattern, shown in FIG. 2, extending in a plurality of directions from the central shaft 58 and extending longitudinally from the first end 54 to the second end 57 of the tank 50. As shown in FIG. 3, each of the rotating members 56 extend radially from the shaft 58 to about the wall 62 of the tank 50, although some clearance may be provided between the wall 62 and the first end 59 of the rotating member 56. The rotating member 56 may further include an arm 61, a folding and scraping paddle 64, and a hook 66. The hook 66 may be located on the arm 61 between the shaft 58 and the paddle 64. The hook 66 is adapted to mix and fold the cellular matter 30 in a different direction than the paddle 64, thereby enhancing the mixing of the cellular matter 30 in a plurality of directions.

In an alternative embodiment of the present invention, the rotating member 56 may be a ribbon-type mixer wherein a plurality of arms are connected by at least one ribbon-like band. Any type of rotating member for mixing known to one of skill in the art may be used to mix the cellular matter 30.

In addition, the number of rotations per minute (rpm) of the center shaft 58 may vary and are adjustable as described above. The cellular matter may also settle by gravity in the tank 50 as well as being dispersed mechanically by the rotating member 56. A portion of the cellular matter 30 mixes within a zone and a portion of the cellular matter 30 moves axially toward the second end 57 of the tank 50. The motion of the rotating member is described above.

In a preferred embodiment, the cellular matter 30 enters the inlet 52 of the bioreactor 20 into the hydrolysis zone 22. In the hydrolysis zone 22, the cellular matter 30 is subjected to sonication to disrupt the cellular matter 30. Sonication of the cellular matter 30 generally begins at the first end 54 of the tank 50 in the hydrolysis zone 22.

In the preferred embodiment shown in FIGS. 2 & 3, the sonic energy is transmitted through the tank wall 62 along the length of a contact plate 70. The contact plate 70 is mounted on the exterior of the wall 62 of the tank 50. A plurality of contact plates 70 may be used to transmit the sonic energy to the cellular matter 30. As shown in FIG. 3, the plurality of cellular plates may be located in the lower half 72 of the tank 50. Although one skilled in the art will recognize that additional arrangements for the contact plate 70 may be acceptable. In a preferred embodiment, the contact plate 70 may be formed from titanium and brazed to the exterior of the wall 62. Any gap between the contact plate and the wall 62 may be filled with a sealant. Other materials and methods known to one of skill in the art may be used to form and attach the contact plate 70 to the tank 50. The number, thickness, width, and length of the contact plate 70 may vary depending on the cellular matter 30 and the desired resulting product 40. Alternatively, the contact plate 70 of the sonic energy source, or any type of horn for transmitting sonic energy to the cellular matter 30 in the tank 50, may be located within the bioreactor 20 itself.

As shown in FIG. 3, a transducer 74 connects to the contact plate 70. A power supply 76 connects to a waveform generator 78 and the waveform generator 78 connects to the transducer 74. The transducer 74 converts the power from the power supply 76 into vibrational acoustic energy that is transmitted through the contact plate 70 and the wall 62 into the tank 50 to disrupt the cellular matter 30 contained therein. Alternatively, a plurality of transducers 74 may be connected to the contact plate 70. A zone of sonication-enhanced degradation 80 is formed within the hydrolysis zone 22 of the tank 50. In the zone 80, the cellular matter 30, being mixed by the at least one rotating member 56, passes in close proximity to the contact plate 70 which is emitting sonic energy. Within the zone 80, microbubbles are formed in the cellular matter 30, leading to cavitation. The energy released during cavitation may be predominantly physical in nature, such as shock waves, hydro-jets, and hydro shear. The cavitation causes the cellular matter 30 passing through the zone 80 to be broken into small pieces. The cavitation process occurs throughout the zone 80 and continues to cause the breakdown of cellular matter 30 as the cellular matter is mixed, folded, and advanced in the bioreactor 20. The process of sonication is repeated the entire length of the contact plate 70 and the cellular matter 30 is repeatedly exposed to the sonic energy in the zone 80 as the rotating member 56 moves the cellular matter 30 in multiple directions within the tank 50. In general, the cellular matter advances toward the second end 57 of the tank 50. Repeated exposure of the cellular matter 30 to the zone 80 provides opportunity to break down essentially the entire volume of the cellular matter 30 as the cellular matter 30 advances in the tank 50. In a preferred embodiment of the present invention, the hydraulic retention time may be reduced from about 20 days, without sonication, to about 5 days with sonication. When a plurality of transducers 74 are connected to the contact plate 70, additional zones of sonic disruption are created. For example, a zone 83 may be created in the acid zone 24 by supplying sonic energy to the tank 50. The supply of the sonic energy is the same as described in the zone 80. The frequency range for the sonic energy supplied to the zone 83 may be in the range of about 1 kHz to about 2,000 kHz. A zone 81 may be created in the methane zone 26 similar to the zone 83. An additional zone 87 may be created in the methane zone 26 near the second end 57 of the tank 50 wherein sonic energy is supplied to the zone 87 in the frequency range of about 1 kHz to about 10 kHz.

When the power in the power supply 76 is activated, the transducer 70 converts the power into vibrational acoustic energy that is then transmitted to the zone 80 as described above. When a plurality of transducers 74 convert the power form the power supply 76 into a plurality of vibrational acoustic energies sonic energy may be provided in a plurality of frequency ranges as described above. The power may be supplied as a continuous supply or alternatively, the power may be an intermittent supply. For example, intermittent power may be supplied to the zone 80 between intermittent rotations of the rotating member 56. The rotating member 56 may mix the cellular matter 30 by rotating on the shaft 58 approximately ⅓ to ¼ turn. When the rotating member 56 stops, the intermittent power supply provides power to the transducer 70 to supply sonic energy to the zone 80 through the contact plate 70 attached to the wall 62. When the power supply is discontinued, the rotating member 56 then rotates the cellular matter 30 another ⅓ to ¼ turn and the cycle is repeated. Alternatively, the sonic energy supplied to the cellular matter 30 may be pulsed with respect to time, wherein the energy is turned off and on, in repetitive cycles without respect to the rotation of the rotating member 56. Alternative mixing and sonic energy supply cycles may be used, as well as continual mixing and/or sonic energy supply. In a preferred embodiment of the present invention, the sonic energy supplied to the zone 80 is in the frequency range from about 1 kHz to about 10 kHz, more preferably in the frequency range from about 2 kHz to about 7 kHz.

After passing through the zone 80, the cellular matter 30 begins to be broken down from large polymers to eventually form smaller subunits. The subunits may begin to be broken down by acid-forming microbes as soon as the subunits are formed. Thus, the subunits may begin to be broken down in the hydrolysis zone 22 as well as in the acid zone 24. Acid-forming microbes are commonly found in the cellular matter 30. As more subunits become available, the acid-forming microbes present in the cellular matter 30 multiply so that the acid zone becomes conditioned with a higher density of microbes in the portion of the acid zone 24 in an acid-conditioned zone 82, toward the second end 57 of the tank 50. In an embodiment, a portion of the conditioned cellular matter 30 having an increased density of acid-forming microbes may be added back to the newly added cellular matter near the beginning of the acid zone 24 into a zone 84. In order to supply conditioned cellular matter 30 to the zone 84, a partial septum 85 and a well 86 may be formed in the wall 62 at the bottom of the tank 50. The septum 85 and the well 86 are positioned in the wall 62 to avoid interference with the rotating member 56. The well 86 collects liquid containing the acid-forming microbes from the acid conditioned zone 82. A removable screen, not shown, may be provided in the well 86 to prevent particulate matter from being collected in the well 86. The liquid from the acid-conditioned zone 82 is fed into an acid zone inlet 88 near the beginning of the zone 84 at the top of the tank 50 to supply a high-density population of acid-forming microbes to further enhance digestion of the cellular matter 30. As described above, the smaller subunits are hydrolyzed by acid-forming microbes to form acetate, long-chain fatty acids, carbon dioxide, H₂, NH₂, and HS⁻. Acetogenic microbes when present may degrade propionate, long-chain fatty acids, alcohols, amino acids, and aromatic compounds to H₂, and acetate. As described above in the hydrolysis zone 22, the rotating member 56 mixes, folds, and advances the cellular matter in the zone 24.

After hydrolysis of the cellular matter 30 in the acid zone 24, the cellular matter 30 advances to the methane zone 26. In an embodiment, the methane zone 26 comprises about 50% to about 67% of the total tank capacity. In the methane zone 26, methanogenic microbes present in the cellular matter 30 further degrades the products formed in the acid zone 24 to produce CO₂ and/or CH₄. Hydrogenotrophic methanogens degrade H₂ to reduce CO₂ to CH₄ and acetotrophic methanogens degrade acetate to form CO₂ and CH₄. Similar to the acid zone 24, the methane zone 26 collects and supplies conditioned cellular matter 30. A septum 89 and a well 90 may be formed in the wall 62 of the tank 50 in the methane zone 26. The septum 89 and the well 90 are positioned in the wall 62 to avoid interference with the rotating member 56. A removable screen, not shown, may be provided in the well 90 to prevent particulate matter from being collected in the well 90. The liquid collected in the well 90 from a methane conditioned zone 92 is fed into a methane zone inlet 94 at the top of the tank 50 near the beginning 96 of the methane zone 26. The liquid containing the high-density conditioned methanogenic microbes further enhances the degradation of the cellular matter 30. As described above for the hydrolysis zone 22 and the acid zone 24, the rotating member 56 mixes, folds, and advances the cellular matter 30 in the zone 26. The tank 50 may further comprise additional inlets, septa, and wells.

Sonication raises the temperature of the liquid medium. The influent cellular matter 30 will not be pre-heated through a heat exchanger as is done in most conventional anaerobic digesters. Temperature surpassing thermophilic range (50-60° C.) can be obtained though sonication. The tank 50 may be heated with a heat source on the exterior surface (not shown) of the tank 50 in the methane zone 26. The bioreactor 20 may be operated as a mesophilic digester with an operating temperature in the range of about 30 to 40° C., or as a thermophilic digester with an operating temperature of about 50-60° C.

Mixing and folding the cellular matter 30 throughout the digestion process will assist in release of biogas and liquid. Biogas will migrate to a headspace 102 and be exhausted through a gas exhaust valve 104 into a gas storage tank (not shown). A slight vacuum will be maintained in the headspace using a pump (not shown), connected to the headspace 102, that exhausts the biogas into the storage tank. The slight vacuum created by drawing off the biogas into the storage tank enhances biogas release from the digested cellular matter 30 due to lower partial pressure that induces gas release from liquid. As described above, the liquid will flow by gravity and accumulate in the wells 86 and 90. The well 86 will collect draining liquid from the acid zone 24, and the well 90 will collect the liquid draining from the methane zone 26. The wells 86 and 90 will be screened with fine stainless steel mesh. Liquid that passes through the screens will be collected and recycled to the head end of acid 24 or methane 26 forming zones. Returning liquids that are biologically active is a common practice in biological waste treatment. Returning liquids accelerates the anaerobic digestion process by introducing active microbial mass at the start of respective acid 24 or methane 26 forming zones.

The level of the cellular matter 30 in the tank 50 is monitored and controlled to maintain a level 106. The level 106 allows room for biogas to accumulate in the headspace 104 and be removed. Nongas products may normally be released from the tank 50 through an outlet 108. Inorganic materials and debris that accumulate in the tank 50 may be intermittently removed from the tank 50 through an outlet 112. The level 106 may be monitored and controlled by any mechanism commonly known to one of skill in the art.

Control of the bioreactor 20 may be manual or automated. In an embodiment of the present invention, the operation of the bioreactor 20 may be automated using a programmable logic controller 110, shown in FIG. 2. The controller 110 may receive and transmit electrical signals from the bioreactor 20 and or remote devices known to one of skill in the art. The controller 110 may monitor and sense information including, but not limited to pH, temperature, pressure, gas flow, and shaft speed. Based on the signals received from the sensors, the controller 110 may automatically implement changes in the operation of the bioreactor 20. Alternatively, the signals received from sensors may be transmitted to a remote receiver for display and manual control.

An alternative embodiment of the present invention is shown in FIG. 5. Numbers for like elements in FIG. 2 have been raised by 200. FIG. 5 shows a bioreactor 220 having an elongated tank 250. The cross sectional view along plane A for the bioreactor 220 is also shown in FIG. 3 and described above.

The bioreactor 250 is adapted to sonically enhance degradation of the cellular matter 30. The cellular matter 30 is subjected to sonic energy in the bioreactor 250. Following treatment in the bioreactor 220, the cellular matter 30 may then be subjected to subsequent treatment, such as anaerobic digestion. The tank 250 is dimensioned similar to the tank 50 shown in FIG. 2. The tank 250 further comprises an inlet 252 into which cellular matter 30 is added to the tank 250. The cellular matter 30 is mixed, folded and advanced by a rotating member 256 attached to a central shaft 258. The shaft 258 extends from a first end 254 to a second end 257 of the tank 250. The shaft 258 is driven by an electric motor 260 having a variable frequency drive. The motor 260 may also provide a constant frequency drive to the shaft 258.

A plurality of rotating members 256 may be used in the tank 250 to mix fold, and advance the cellular matter 30 from the first end 254 to the second end 257. In the tank 250, the cellular matter 30 is subjected to sonication to disrupt the cellular matter 30. The sonic energy may be transmitted through the tank wall 262. A contact plate 270 is mounted to the exterior of the tank 250 and may extend a majority of the length of the tank 252. A plurality of contact plates 270 may be used to transmit sonic energy to the cellular matter 30 within the tank 250. The mounting of the contact plate 270 and the transmission of sonic energy and the components used therefore are the same as describe above for the contact plate 70 and the tank 50. The power supplied to the plate 270 is the same as the power supplied to the plate 70 in the tank 50 and is shown in FIG. 3 and described above.

A zone of sonication-enhanced degradation 280 is formed within the tank 250. The zone 280 parallels the contact plate 270 and is formed in the tank 250. In the zone 280, the cellular matter 30 being mixed by the rotating member 256 passes in close proximity to the contact plate 70 that is emitting sonic energy. In an embodiment of the present invention, the sonic energy supplied to the zone 280 is in the frequency range from about 1 kHz to about 10 kHz, more preferably from about 2 kHz to about 7 kHz.

A level 306 of the cellular matter 30 within the tank 250 is monitored and controlled as described above for the tank 50.

After the cellular matter 30 has been subjected to sonication in the zone 280, the sonicated cellular matter 30 may then be removed from the tank 250 through an outlet 290. Inorganic materials and debris that accumulate in tank 250 may be intermittently removed from tank 250 through an outlet 292. The cellular matter is subjected to sonication in the bioreactor 220 for a time sufficient to disintegrate suspended solid particles in the cellular matter as well as disrupt and breakdown the cellular matter into smaller subunits. The cellular matter 30 may then be transferred to at least one more bioreactor 20. The at least one more bioreactor 20 may comprise, but are not limited to, an acid zone 24 and a methane zone 26. The acid zone 24 and the methane zone 26 may be in one bioreactor 20, or alternatively, each zone may be in a separate bioreactor. The additional bioreactors in the embodiment comprising multiple bioreactor function like the similar zones described above for the single bioreactor.

Although the invention herein has been described in connection with an embodiment thereof, it will be appreciated by those skilled in the art that additions, modifications, substitutions, and deletions not specifically described may be made without departing from the spirit and scope of the invention as defined in the appended claims. It is therefore intended that the foregoing detailed description be regarded as illustrative rather than limiting, and that it be understood that it is the following claims, including all equivalents, that are intended to define the spirit and scope of this invention.

1. A method for sonication-enhanced digestion of cellular matter, said method comprising:
supplying cellular matter comprising microbes to a bioreactor;
sonically disrupting said cellular matter; and
mixing the cellular matter in the bioreactor for a time sufficient for the cellular matter to be digested by the microbes.

2. The method of claim 1 wherein sonically disrupting said cellular matter comprises supplying sonic energy to a first zone of said bioreactor in the frequency range of about 1 kHz to about 10 kHz.

3. The method of claim 1 wherein sonically disrupting said cellular matter comprises supplying sonic energy to a plurality of zones.

4. The method of claim 3 comprising supplying sonic energy to a first zone in a frequency range of about 1 KHz to about 10 kHz.

5. The method of claim 3 comprising supplying sonic energy to a second zone in a frequency range of about 1 KHz to about 2,000 kHz.

6. The method of claim 1 wherein said mixing of said cellular matter is at about 0.25 rpm to about 5.0 rpm.

7. The method of claim 1 further comprising supplying conditioned microbes to said bioreactor.

8. The method of claim 7 wherein said microbes are acid-forming microbes.

9. The method of claim 7 wherein said microbes are methanogenic microbes.

10. The method of claim 1 wherein a vacuum is provided within the bioreactor.

11. The method of claim 1 further comprising removing biogas produced in said bioreactor.

12. The method of claim 1 further comprising heating of a portion of said bioreactor.

13. The method of claim 1 further comprising supplying a process controller to monitor and modify said sonic disruption and providing said disrupted cellular material for microbial production.

14. The method of claim 1 wherein said cellular matter is manure.

15. The method of claim 1 wherein said cellular matter is lignocellulose.

16. The method of claim 1 wherein said cellular matter is municipal wastes or sludge.

17. The method of claim 1 wherein said cellular matter is industrial wastes or sludge.

18. A method for sonication-enhanced degradation of cellular matter, said method comprising:
supplying cellular matter comprising microbes to a first bioreactor; and
subjecting said cellular matter to sonic energy in a frequency range of about 1 kHz to about 10 kHz in said first bioreactor.

19. The method of claim 18 further comprising mixing said cellular matter in said first bioreactor.

20. The method of claim 18 wherein said mixing of said cellular matter is at about 0.25 rpm to about 5.0 rpm.

21. The method of claim 18 further comprising supplying a process controller to monitor and modify said sonic disruption.

22. The method of claim 18 wherein said cellular matter is manure.

23. The method of claim 18 wherein said cellular matter is lignocellulose.

24. The method of claim 18 wherein said cellular matter is municipal wastes or sludge.

25. The method of claim 18 wherein said cellular matter is industrial wastes or sludge.

26. The method of claim 18 further comprising supplying said cellular matter from said first bioreactor to a second bioreactor.

27. The method of claim 26 further comprising subjecting said cellular matter to sonic energy in a frequency range of about 1 kHz to about 2,000 kHz.

28. The method of claim 26 further comprising mixing said cellular matter in said bioreactor.

29. The method of claim 28 wherein said mixing of said cellular matter is at about 0.25 rpm to about 5.0 rpm.

30. The method of claim 26 further comprising supplying conditioned microbes to said second bioreactor.

31. The method of claim 30 wherein said microbes are acid-forming microbes.

32. The method of claim 30 wherein said microbes are methanogenic microbes.

33. The method of claim 26 wherein a vacuum is provided within said second bioreactor.

34. The method of claim 26 further comprising removing biogas produced in said second bioreactor.

35. The method of claim 26 further comprising heating of a portion of said bioreactor.

36. The method of claim 26 further comprising supplying a process controller to monitor and modify said sonic disruption and providing said disrupted cellular material for microbial production.

37. A system for sonication-enhanced digestion of cellular matter, said system comprising:
a bioreactor for cellular matter, said bioreactor having an inlet and an outlet;
a sonic energy source operatively connected to said bioreactor; and
at least one rotating member operatively connected to said bioreactor;
wherein cellular matter enters said bioreactor through said inlet, is mixed by said at least one rotating member and is subjected to sonic energy and microbial digestion in said bioreactor.

38. The system of claim 37 wherein said sonic energy source supplies sonic energy to said cellular matter in a first zone of said bioreactor in a frequency range of about 1 kHz to about 10 kHz.

39. The system of claim 37 wherein said sonic energy source supplies sonic energy to said cellular matter in a plurality of zones.

40. The system of claim 39 wherein said sonic energy source supplies sonic energy to said cellular matter in a first zone in a frequency range of about 1 KHz to about 10 kHz.

41. The system of claim 39 wherein said sonic energy source supplies sonic energy to said cellular matter in a second zone in a frequency range of about 1 KHz to about 2,000 kHz.

42. The system of claim 37 wherein said sonic energy source comprises a contact plate operatively connected to said bioreactor, a transducer operatively connected to said contact plate, a wave-form generator operatively connected to said transducer and a power supply operatively connected to said wave-form generator.

43. The system of claim 37 wherein a vacuum is maintained in said bioreactor.

44. The system of claim 37 further comprising microbes supplied to said bioreactor.

45. The system of claim 44 wherein said microbes are acid-forming microbes.

46. The system of claim 44 wherein said microbes are methanogenic microbes.

47. The system of claim 37 wherein the bioreactor has a first end and a second end and wherein said second end is elevated with respect to said first end.

48. The system of claim 37 further comprising a means for heating said a portion of said bioreactor.

49. The system of claim 37 further comprising a gas exhaust valve operatively connected to said bioreactor.

50. The system of claim 37 further comprising a process controller to monitor and modify said sonic energy supply and said microbial digestion.

51. An apparatus for sonication-enhanced degradation of cellular matter, said apparatus comprising:
a first bioreactor; and
a first sonic energy source operatively connected to said first bioreactor; said sonic energy source subjects cellular matter to sonic energy in a frequency range of about 1 kHz to about 10 kHz.

52. The apparatus of claim 51 further comprising at least one rotating member operatively connected to said first bioreactor.

53. The apparatus of claim 51 wherein said sonic energy source comprises a contact plate operatively connected to said first bioreactor, a transducer operatively connected to said contact plate, a wave-form generator operatively connected to said transducer and a power supply operatively connected to said transducer.

54. The apparatus of claim 51 further comprising a process controller to monitor and modify said sonic energy supply.

55. The apparatus of claim 51 further comprising a second bioreactor operatively connected to said first bioreactor.

56. The apparatus of claim 54 further comprising a second sonic energy source operatively connected to said second bioreactor; said second sonic energy source subjects cellular matter to sonic energy in a frequency range of about 1 kHz to about 2,000 kHz.

57. The apparatus of claim 55 wherein said second sonic energy source comprises a contact plate operatively connected to said second bioreactor, a transducer operatively connected to said contact plate, a wave-form generator operatively connected to said transducer and a power supply operatively connected to said transducer.

58. The apparatus of claim 53 further comprising at least one rotating member.

59. The apparatus of claim 55 further comprising a process controller.

60. The apparatus of claim 55 further comprising a third bioreactor operative connected to said second bioreactor.

61. A system for sonication-enhanced digestion of cellular matter, said system comprising:
a bioreactor for cellular matter;
means for sonically disrupting cellular matter contained in said bioreactor; and
means for mixing said cellular matter in said bioreactor;
wherein cellular matter is sonically disrupted, mixed, and digested in said bioreactor.
